(19) United States
(12) Patent Application Publication
(10) Pub. No.: US 2005/0201974 A1
(43) Pub. Date: Sep. 15, 2005

(54) Bioadhesive polymers with catechol functionality

(75) Inventors: Marcus Schestopol, Providence, RI (US); Jules Jacob, Taunton, MA (US); Ryan Donnelly, Quincy, MA (US); Thomas Ricketts, Halifax, MA (US); Avinash Nangia, Wrentham, MA (US); Edith Mathiowitz, Brookline, MA (US); Ze'ev Shaked, San Antonio, TX (US)

Correspondence Address: PATREA L. PABST;PABST PATENT GROUP LLP, 400 COLONY SQUARE, SUITE 1200, ATLANTA, GA, 30361, US

(73) Assignee: SPHERICS, INC.

(21) Appl. No.: 11/009,327

(22) Filed: Dec. 9, 2004

Related U.S. Application Data
(60) Provisional application No. 60/528,042, filed on Dec. 9, 2003.
(60) Provisional application No. 60/605,201, filed on Aug. 27, 2004.
(60) Provisional application No. 60/605,199, filed on Aug. 27, 2004.
(60) Provisional application No. 60/607,905, filed on Sep. 8, 2004.
(60) Provisional application No. 60/604,990, filed on Aug. 27, 2004.

(51) Int. Cl. A61K 31/74

(52) U.S. Cl. 424/78.27

(57) ABSTRACT

Polymers with improved bioadhesive properties and methods for improving bioadhesion of polymers have been developed. A compound containing an aromatic group which contains one or more hydroxyl groups is grafted onto a polymer or coupled to individual monomers. In one embodiment, the polymer is a biodegradable polymer. In another embodiment, the monomers may be polymerized to form any type of polymer, including biodegradable and non-biodegradable polymers. In some embodiments, the polymer is a hydrophobic polymer. In the preferred embodiment, the aromatic compound is catechol or a derivative thereof and the polymer contains reactive functional groups. In the most preferred embodiment, the polymer is a polyanhydride and the aromatic compound is the catechol derivative, DOPA. These materials display bioadhesive properties superior to conventional bioadhesives used in therapeutic and diagnostic applications. These bioadhesive materials can be used to fabricate new drug delivery or diagnostic systems with increased residence time at tissue surfaces, and consequently increase the bioavailability of a drug or a diagnostic agent. In a preferred embodiment, the bioadhesive material is a coating on a controlled release oral dosage formulation and/or forms a matrix in an oral dosage formulation.

### CROSS-REFERENCE TO RELATED APPLICATIONS

This application claims priority to U.S. Ser. No. 60/528,042, entitled “Bioadhesive Polymers with Catechol Functionality” to Marcus A Schestopol and Jules S. Jacob, filed Dec. 9, 2003. This application also claims priority to U.S. Ser. No. 60/605,201, entitled “Mucoadhesive Oral Formulations of High Permeability, Low Solubility Drugs”, filed Aug. 27, 2004; U.S. Ser. No. 60/605,199, entitled “Mucoadhesive Oral Formulations of Low Permeability, Low Solubility Drugs”, filed Aug. 27, 2004; U.S. Ser. No. 60/604,990, entitled “Bioadhesive Rate Controlled Oral Dosage Formulation”, filed Aug. 27, 2004; and U.S. Ser. No. 60/607,905, entitled “Mucoadhesive Oral Formulations of High Permeability, High Solubility Drugs”, filed Sep. 8, 2004.

### FIELD OF THE INVENTION

The present invention relates to polymers with improved bioadhesion and methods for improving the bioadhesion of polymers.

### BACKGROUND OF THE INVENTION

Polymers that adhere well to biological surfaces (“bioadhesives”) under a variety of conditions are useful in several branches of medicine. One important use of bioadhesive polymers is in drug delivery systems, particularly oral drug delivery. Such bioadhesive polymers, for example, certain polyanhydrides, are useful for slowing the passage of drug-containing materials through the gastrointestinal tract. U.S. Pat. No. 6,197,346 to Mathiowitz et al. describes using bioadhesive polymers that have high concentrations of carboxylic acid groups, such as polyanhydrides, to form microcapsules or as a coating on microcapsules which contain therapeutic or diagnostic agents.

Polyanhydrides are bioadhesive in vivo, for example in the gastrointestinal (GI) tract, and can significantly delay the passage of drug-containing particles through the GI tract, thus allowing more time for absorption of drug by the intestine. The mechanism causing the anhydride polymers or oligomers to be bioadhesive is believed to be due to a combination of the polymer's hydrophobic backbone, coupled with the presence of carboxyl groups at the ends. Interaction of charged carboxylate groups with tissue has been demonstrated with other bioadhesives. In particular, pharmaceutical industry materials considered to be bioadhesive typically are hydrophilic polymers containing carboxylic acid groups, and often hydroxyl groups as well. The industry standard is often considered to be CARBOPOL™ (a high molecular weight poly(acrylic acid)). Other classes of bioadhesive polymers are characterized by having moderate to high densities of carboxyl substitution. The relatively hydrophobic anhydride polymers frequently demonstrate superior bioadhesive properties when compared with the hydrophilic carboxylate polymers. However, all of these polymer adhesives tend to lose effectiveness when wet, and especially when wetting is prolonged. Their reduced adhesion to surfaces in vivo tends to diminish their effectiveness in enhancing drug delivery.

Natural adhesives for underwater attachment of mussels, other bivalves and algae to rocks and other substrates are known (see U.S. Pat. No. 5,574,134 to Waite, U.S. Pat. No. 5,015,677 to Benedict et al., and U.S. Pat. No. 5,520,727 to Vreeland et al.). These adhesives are polymers containing poly(hydroxy-substituted) aromatic groups. In mussels and other bivalves, such polymers include dihydroxy-substituted aromatic groups, such as proteins containing 3,4-dihydroxyphenylalanine (DOPA). In algae, diverse polyhydroxy aromatics such as phloroglucinol and tannins are used. In adhering to an underwater surface, the bivalves secrete a preformed protein that adheres to the substrate thereby linking the bivalve to the substrate. After an initial adherence step, the natural polymers are typically permanently crosslinked by oxidation of adjacent hydroxyl groups.

Extraction of these materials from organisms is not practical for commercial scale production. Attempts to reproduce the adherence have been made, typically using synthetic or genetically engineered polypeptides containing amino acid motifs derived from mussel adhesives, or natural marine materials. The synthetic protein materials have proved to be too expensive, or otherwise inadequate, to sustain commercial applications. The need for an enzyme-mediated oxidation state of the hydroxyl groups on the polymers is an additional barrier to use.

Earlier approaches to adhesive polymers, such as U.S. Pat. No. 4,908,404 to Benedict et al., require grafting DOPA to polyamines. However, the adhesiveness of these cationic water-soluble compounds is not much better than that of the parent polyamines, such as poly-L-lysine.

Therefore it is an object of the invention to provide polymers with improved bioadhesive properties, particularly when the polymers and/or the surfaces are wet.

It is a further object of the invention to provide a method for improving the bioadhesive properties of polymers.

It is a still further object of the invention to provide drug delivery systems with increased residence times in the GI tract, nasal mucosa, pulmonary mucosa, and other mucosa in a cost-effective manner.

### BRIEF SUMMARY OF THE INVENTION

Polymers with improved bioadhesive properties and methods for improving bioadhesion of polymers have been developed. A compound containing an aromatic group which contains one or more hydroxyl groups is grafted onto a polymer or coupled to individual monomers. In one embodiment, the polymer is a biodegradable polymer. In another embodiment, the monomers may be polymerized to form any type of polymer, including biodegradable and non-biodegradable polymers. In some embodiments, the polymer is a hydrophobic polymer. In the preferred embodiment, the aromatic compound is catechol or a derivative thereof and the polymer contains reactive functional groups. In the most preferred embodiment, the polymer is a polyanhydride and the aromatic compound is the catechol derivative, DOPA. These materials display bioadhesive properties superior to conventional bioadhesives used in therapeutic and diagnostic applications. These bioadhesive materials can be used to fabricate new drug delivery or diagnostic systems with increased residence time at tissue surfaces, and consequently increase the bioavailability of a drug or a diagnostic agent. In a preferred embodiment, the bioadhesive material is a coating on a controlled release oral dosage formulation and/or forms a matrix in an oral dosage formulation.

### BRIEF DESCRIPTION OF THE DRAWINGS

FIG. 1 is a bar graph showing the fracture strength of bonds (mN/cm²) formed with the bioadhesive materials (poly(butadiene maleic anhydride copolymer)-DOPA) as compared to controls (poly(butadiene maleic anhydride copolymer)).

FIG. 2 is a bar graph of the tensile work (nJ) required to rupture the bonds formed with the bioadhesive materials (poly(butadiene maleic anhydride copolymer)-DOPA) as compared to controls (poly(butadiene maleic anhydride copolymer)).

FIG. 3 is a cross-section of a bioadhesive rate-controlling oral dosage formulation (BIOROD).

FIG. 4 is a cross-section of a BIOROD containing multiparticulates.

FIG. 5 is a cross-section of a BIOROD with restricted release openings.

FIG. 6 is a cross-section of a BIOROD with multiple drug layers and restricted release openings.

FIG. 7 is a cross-section of an osmotic BIOROD system.

FIG. 8 is a cross-section of a push-pull osmotic BIOROD system.

FIG. 9 is a cross-section of a push-pull osmotic BIOROD system with an insoluble plug between the drug layer and the polymer layer.

FIG. 10 is a cross-section of a push-pull osmotic BIOROD system with an insoluble plug beneath the polymer layer.

FIG. 11 is a cross-section of a two-pulse BIOROD system.

FIG. 12 is a cross-section of a tablet containing precompressed inserts of an active agent.

FIG. 13 is a graph which shows a plasma itraconazole pharmacokinetic profile of controlled release itraconazole oral dosage formulation dosed to fed beagle dogs (n=6/test).

FIG. 14 is a graph showing a comparison of AUC, Cmax, and Tmax values of Tablet 1 (bioadhesive controlled release formulation) and ZOVIRAX® 400 mg tablet (Immediate Release formulation).

FIG. 15 is a graph showing a comparison of AUC, Cmax, and Tmax values of Tablet 2 (bioadhesive controlled release formulation) and ZOVIRAX® 400 mg tablet (Immediate Release formulation).

### DETAILED DESCRIPTION OF THE INVENTION

I. Bioadhesives

As generally used herein “bioadhesives” or “bioadhesive materials” refer to the polymers which are modified to have improved bioadhesion.

As used herein “bioadhesion” generally refers to the ability of a material to adhere to a biological surface for an extended period of time. Bioadhesion requires a contact between the bioadhesive material and the receptor surface, the bioadhesive material penetrates into the crevice of the surface (e.g. tissue and/or mucus) and chemical bonds form. Thus the amount of bioadhesive force is affected by both the nature of the bioadhesive material, such as a polymer, and the nature of the surrounding medium. Adhesion of polymers to tissues may be achieved by (i) physical or mechanical bonds, (ii) primary or covalent chemical bonds, and/or (iii) secondary chemical bonds (i.e., ionic). Physical or mechanical bonds can result from deposition and inclusion of the adhesive material in the crevices of the mucus or the folds of the mucosa. Secondary chemical bonds, contributing to bioadhesive properties, consist of dispersive interactions (i.e., Van der Waals interactions) and stronger specific interactions, which include hydrogen bonds. The hydrophilic functional groups responsible for forming hydrogen bonds are the hydroxyl (—OH) and the carboxylic groups (—COOH). Bioadhesive forces are measured in units of N/m², by methods defined in U.S. Pat. No. 6,197,346 to Mathiowitz et al., which is herein incorporated by reference. Bioadhesive forces, especially those exhibited by tablets, can also be measured using a Texture Analyser, such as the TA-TX2 Texture Analyser (Stable Micro Systems, Haslemer, Surrey, UK). As described in Michael J. Tobyn et al, Eur. J. Pharm. Biopharm., 41(4): 235-241 (1995), a mucoadhesive tablet is attached to a probe on the texture analyzer and lowered until it contacts pig gastric tissue, which is attached to a tissue holder and exposed to liquid at 37° C. to simulate gastric medium. A force is applied for a set period of time and then the probe is lifted at a set rate. Area under the force/distance curve calculations are used to determine the work of adhesion.(See also Michael J. Tobyn et al., Eur. J. Pharm. Biopharm., 42(1): 56-61 (1996) and David S. Jones, et al., International J Pharmaceutics, 151: 223-233 (1997)).

As used herein “catechol” refers to a compound with a molecular formula of C₆H₆O₂ and the following structure:

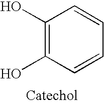

Bioadhesive materials contain a polymer with a catechol functionality. The molecular weight of the bioadhesive materials and percent substitution of the polymer with the aromatic compound may vary greatly. The degree of substitution varies based on the desired adhesive strength, it may be as low as 10%, 20%, 25%, 50%, or up to 100% substitution. On average at least 50% of the monomers in the polymeric backbone are substituted with at least one aromatic group. Preferably, 75-95% of the monomers in the backbone are substituted with at least one aromatic group or a side chain containing an aromatic group. In the preferred embodiment, on average 100% of the monomers in the polymeric backbone are substituted with at least one aromatic group or a side chain containing an aromatic group. The resulting bioadhesive material is a polymer with a molecular weight ranging from about 1 to 2,000 kDa.

a. Polymers

The polymer that forms that backbone of the bioadhesive material may be any non-biodegradable or biodegradable polymer. In the preferred embodiment, the polymer is a hydrophobic polymer. In one embodiment, the polymer is a biodegradable polymer and is used to form an oral dosage formulation.

Examples of preferred biodegradable polymers include synthetic polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers.

In one embodiment, the polymer is formed by first coupling the aromatic compound to the monomer and then polymerizing. In this embodiment, the monomers may be polymerized to form any polymer, including biodegradable and non-biodegradable polymers. Suitable polymers include, but are not limited to: polyanhydrides, polyamides, polycarbonates, polyalkylenes, polyalkylene oxides such as polyethylene glycol, polyalkylene terepthalates such as poly(ethylene terephthalate), polyvinyl alcohols, polyvinyl ethers, polyvinyl esters, polyethylene, polypropylene, poly(vinyl acetate), poly vinyl chloride, polystyrene, polyvinyl halides, polyvinylpyrrolidone, polyhydroxy acids, polysiloxanes, polyurethanes and copolymers thereof, modified celluloses, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate).

The polymer may be a known bioadhesive polymer that is hydrophilic or hydrophobic. Hydrophilic polymers include CARBOPOL™ (a high molecular weight, crosslinked, acrylic acid-based polymers manufactured by NOVEON™), polycarbophil, cellulose esters, and dextran.

In some embodiments, one can use non-biodegradable polymers, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

Hydrophobic polymers include polyanhydrides, poly(ortho)esters, and polyesters such as polycaprolactone. In the preferred embodiment, the polymer is sufficiently hydrophobic that it is not readily water soluble, for example the polymer should be soluble up to less than about 1% w/w in water, preferably about 0.1% w/w in water at room temperature or body temperature. In the most preferred embodiment, the polymer is a polyanhydride, such as a poly(butadiene maleic anhydride) and other copolymers of maleic anhydrides.

Polyanhydrides may be formed from dicarboxylic acids as described in U.S. Pat. No. 4,757,128 to Domb et al., herein incorporated by reference. Suitable diacids include: aliphatic dicarboxylic acids, aromatic dicarboxylic acids, aromatic-aliphatic dicarboxylic acid, combinations of aromatic, aliphatic and aromatic-aliphatic dicarboxylic acids, aromatic and aliphatic heterocyclic dicarboxylic acids, and aromatic and aliphatic heterocyclic dicarboxylic acids in combination with aliphatic dicarboxylic acids, aromatic-aliphatic dicarboxylic acids, and aromatic dicarboxylic acids of more than one phenyl group. Suitable monomers include sebacic acid (SA), fumaric acid (FA), bis(p-carboxyphenoxy)propane (CPP), isophthalic acid (IPh), and dodecanedioic acid (DD).

A wide range of molecular weights are suitable for the polymer that forms the backbone of the bioadhesive material. The molecular weight may be as low as about 200 Da (for oligomers) up to about 2,000 kDa. Preferably the polymer has a molecular weight of at least 1,000 Da, more preferably at least 2,000 Da, most preferably the polymer has a molecular weight of up to 20 kDa or up to 200 kDa. The molecular weight of the polymer may be up to 2,000 kDa.

The range of substitution on the polymer varies greatly and depends on the polymer used and the desired bioadhesive strength. For example, a butadiene maleic anhydride copolymer that is 100% substituted with DOPA will have the same number of DOPA molecules per chain length as a 67% substituted ethylene maleic anhydride copolymer. Typically, the polymer has a percent substitution ranging from 10% to 100%, preferably greater than 50%, ranging from 50% to 100%.

The polymers and copolymers that form the backbone of the bioadhesive material contain reactive functional groups which interact with the functional groups on the aromatic compound.

b. Reactive Functional Groups

It is important that the polymer or monomer that forms the polymeric backbone contains accessible functional groups that easily react with functional groups contained in the aromatic compounds, such as amines and thiols. In a preferred embodiment, the polymer contains amino reactive moieties, such as aldehydes, ketones, carboxylic acid derivatives, cyclic anhydrides, alkyl halides, acyl azides, isocyanates, isothiocyanates, and succinimidyl esters.

c. Sidechains Containing Aromatic Groups with One or More Hydroxyl Groups

Aromatic groups containing one or more hydroxyl groups are attached to the polymeric backbone. The aromatic groups may be part of a compound that is grafted to the polymer backbone or the aromatic groups may be part of larger sidechains which are grafted to the polymer backbone. In the preferred embodiment, the aromatic group containing one or more hydroxyl groups is catechol or a derivative thereof. Optionally the aromatic compound is a polyhydroxy aromatic compound, such as a trihydroxy aromatic compound (e.g. phloroglucinol) or a multihydroxy aromatic compound (e.g. tannin). The catechol derivative may also contain a reactive group, such as an amino, thiol, or halide group. Suitable sidechains which can be grafted to the polymer backbone include poly (amino acids), peptides, or proteins, having a molecular weight of 20 kDa or less, where at least 10% of the amino acids contain catechol residues. Preferably greater than 50%, more preferably 75%, and most preferably 100% of the amino acids contain catechol residues. Common amino acids with catechol-like residues are phenylanine, tyrosine and tryptophan. Additionally, synthetic amino acids that contain catechol residues may be prepared.

The preferred catechol derivative is 3,4-dihydroxyphenylalanine (DOPA), which contains a primary amine. L-DOPA is known to be pharmaceutically active and is used as a treatment for Parkinson's disease. Tyrosine, the immediate precursor of DOPA, which differs only by the absence of one hydroxyl group in the aromatic ring, can also be used. Tyrosine is capable of conversion (e.g. by hydroxylation) to the DOPA form.

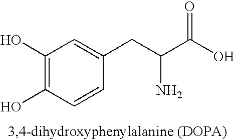

In the preferred embodiment, the aromatic group is an amine-containing aromatic compound, such as an amine-containing catechol derivative.

II. Method of forming Bioadhesives

Two general methods are used to form the bioadhesive materials. In one embodiment, a compound containing an aromatic group which contains one or more hydroxyl groups is grafted onto a polymer. In this embodiment, the polymeric backbone is a biodegradable polymer. In a second embodiment, the aromatic compound may be coupled to individual monomers and then polymerized.

Any chemistry which allows for the conjugation of a polymer or monomer to an aromatic compound containing one or more hydroxyl groups may be used. For example, if the aromatic compound contains an amino group and the monomer or polymer contains an amino reactive group, this modification to the polymer or monomer is performed through a nucleophilic addition or a nucleophilic substitution reaction, including a Michael-type addition reaction, between the amino group in the aromatic compound and the polymer or monomer. Additionally, other procedures can be used in the coupling reaction. For example, carbodiimide and mixed anhydride based procedures form stable amide bonds between carboxylic acids or phosphates and amino groups, bifunctional aldehydes react with primary amino groups, bifunctional active esters react with primary amino groups, and divinylsulfone facilitates reactions with amino, thiol, or hydroxy groups.

a. Polymer Grafting

The aromatic compounds are grafted onto the polymer using standard techniques to form the bioadhesive material. An example of the grafting procedure is schematically depicted in Reaction 1, which depicts a nucleophilic substitution reaction between the amino group in the aromatic compound and the polymer. L-DOPA is grafted to maleic anhydride copolymers by reacting the free amine in L-DOPA with the maleic anhydride bond in the copolymer.

A variety of different polymers can be used as the backbone of the bioadhesive material. Representative polymers include 1:1 random copolymers of maleic anhydride with ethylene, vinyl acetate, styrene, or butadiene. The variable portions of the backbone structures are designated as the R groups at the bottom of Reaction 1. In addition, a number of other compounds containing aromatic rings with hydroxy substituents, such as tyrosine or derivatives of catechol, can be used in reaction 1.

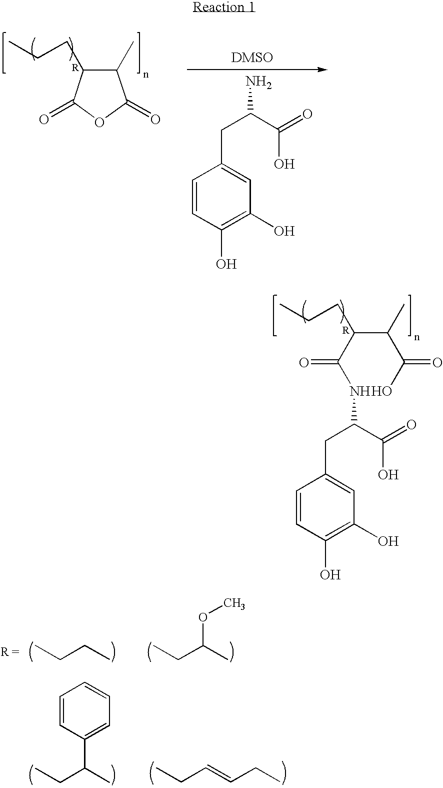

b. Polymer Building

In another embodiment, the polymers are prepared by conjugate addition of a compound containing an aromatic group and an amine functionality to one or more monomers containing an amino reactive group. In the preferred method the monomer is an acrylate or a polymer acrylate. In the most preferred method the monomer is a diacrylate such as 1,4-butanediol diacrylate; 1,3-propanediol diacrylate; 1,2-ethanediol diacrylate; 1,6-hexanediol diacrylate; 2,5-hexanediol diacrylate; or 1,3-propanediol diacrylate. In the coupling reaction, the monomer and the compound containing an aromatic group are each dissolved in an organic solvent (e.g., THF, CH₂Cl₂, MeOH, EtOH, CHCl₃, hexanes, toluene, benzene, CCl₄, glyme, diethyl ether, etc.) to form two solutions. The resulting solutions are combined, and the reaction mixture is heated to yield the desired polymer. The molecular weight of the synthesized polymer may be determined by the reaction conditions (e.g., temperature, starting materials, concentration, solvent, etc) used in the synthesis.

For example, a monomer, such as 1,4 phenylene diacrylate or 1,4 butanediol diacrylate having a concentration of 1.6 M, and DOPA or another primary amine containing aromatic molecule are each dissolved in an aprotic solvent such as DMF or DMSO to form two solutions, the solutions are mixed in a 1:1 molar ratio between the diacrylate and the amine group and heated to 56° C. to form a bioadhesive material.

III. Applications for Bioadhesives

Bioadhesive materials described herein may be used in a wide variety of drug delivery and diagnostic applications. Bioadhesive materials may be formed into microparticles, such as microspheres or microcapsules, or may be a coating on such microparticles. In the preferred embodiment, the material is applied as a coating to a solid oral dosage formulation, such as a tablet or gel-capsule or to multiparticulates. The coating may be applied by direct compression or by applying a solution containing the material to the tablets or gel-capsules. In one embodiment, the bioadhesive material is in the matrix of a tablet or other drug delivery device. Optionally, the tablet or drug delivery device contains a coating, such as a coating containing the bioadhesive material or another bioadhesive polymer or an enteric coating.

In one embodiment, the bioadhesive material is used in drug depot or reservoir systems, such as an osmotic drug delivery system. In this embodiment, the bioadhesive material may be present in a matrix surrounding the drug to be delivered and/or as a coating on the surface of the system. The depot or reservoir systems contain a microporous or macroporous membrane that separates the outside environment from the drug inside the system. The osmotic delivery system contains osmotic agents, which bring water into the system, causing a swellable material, such as a polymeric matrix or separate polymeric layer, to swell. When the material inside the system swells, it pushes the drug against the semi-permeable membrane and out of the system.

The bioadhesive coating adheres to the mucosa in the aqueous environment of the gastrointestinal tract. As a result, the bioavailability of therapeutic agents is enhanced through increased residence time at the target absorption rate. In a preferred embodiment, the solid oral dosage form contains rate controlling agents, such as hydroxypropylmethyl cellulose (HPMC) and microcrystalline cellulose (MCC). Optionally, the drug may be in the form or microparticles or nanoparticles. In one embodiment, a tablet contains a core containing nanoparticulate drug and enhancers in a central matrix of rate controlling agents, such as hydroxypropylmethyl cellulose (HPMC) and microcrystalline cellulose (MCC). The core is surrounded on its circumference by bioadhesive polymer (preferably DOPA-BMA polymer). Optionally, the final tablet is coated with an enteric coating, such as Eudragit L100-55, to prevent release of the drug until the tablet has moved to the small intestine.

The bioadhesive materials may be used in or as a coating on prosthetics, such as dental prosthetics. The materials may be used as dental adhesives, or bone cements and glues. The materials are suitable for use in wound healing applications, such as synthetic skins, wound dressings, and skin plasters and films.

a. Materials that can be Incorporated into the Bioadhesive Materials

There is no specific limitation on the material that can be encapsulated within the bioadhesive materials. Any kind of therapeutic, prophylactic or diagnostic agent, including organic compounds, inorganic compounds, proteins, polysaccharides, nucleic acids, or other materials can be incorporated using standard techniques. Flavorants, nutraceuticals, and dietary supplements are among the materials that can be incorporated in the bioadhesive material. In the preferred embodiment, L-3,4-dihydroxyphenylalanine (“levodopa” or “L-dopa”) is incorporated into the bioadhesive material for delivery to a patient. The bioadhesive material may contain carbidopa. In one embodiment, levodopa and carbidopa are both incorporated in the bioadhesive material. In a preferred embodiment, the bioadhesive material is a coating on an oral dosage formulation which contains levodopa and carbidopa in separate drug layers.

Examples of useful proteins include hormones such as insulin, growth hormones including somatomedins, transforming growth factors and other growth factors, antigens for oral vaccines, enzymes such as lactase or lipases, and digestive aids such as pancreatin.

Examples of useful drugs include ulcer treatments such as Carafate from Marion Pharmaceuticals, antihypertensives or saluretics such as Metolazone from Searle Pharmaceuticals, carbonic anhydrase inhibitors such as Acetazolamide from Lederle Pharmaceuticals, insulin-like drugs such as glyburide, a blood glucose lowering drug of the sulfonylurea class, hormones such as Android F from Brown Pharmaceuticals and Testred (methyltestosterone) from ICN Pharmaceuticals, antiparasitics such as mebeandazole (VERMOX™, Jannsen Pharmaceutical). Other drugs for application to the vaginal lining or other mucosal membrane lined orifices such as the rectum include spermacides, yeast or trichomonas treatments and anti-hemorrhoidal treatments.

Drugs may be classified using the Biopharmaceutical Classification System (BCS), which separates pharmaceuticals for oral administration into four classes depending on their solubility and their absorbability through the intestinal cell layer. According to the BCS, drug substances are classified as follows:
Class I—High Permeability, High Solubility
Class II—High Permeability, Low Solubility
Class III—Low Permeability, High Solubility
Class IV—Low Permeability, Low Solubility

The interest in this classification system stems largely from its application in early drug development and then in the management of product change through its life-cycle. In the early stages of drug development, knowledge of the class of a particular drug is an important factor influencing the decision to continue or stop its development.

Class I drugs of the BCS system are highly soluble and highly permeable in the gastrointestinal (GI) tract. Representative BCS Class I drugs include caffeine, carbamazepine, fluvastatin, Ketoprofen, Metoprolol, Naproxen, Propranolol, Theophylline, Verapamil. Diltiazem, Gabapentin, Levodopa CR, and Divalproex sodium. Sometimes BCS Class I drugs may be micronized to sizes less than 2 microns to increase the rate of dissolution. Other means to micronize or molecularly disperse drugs in a polymer matrix include spray-drying, drug-layering, hot-melt extrusion, and super-critical fluid micronization.

Class II drugs are drugs that are particularly insoluble, or slow to dissolve, but that readily are absorbed from solution by the lining of the stomach and/or the intestine. Hence, prolonged exposure to the lining of the GI tract is required to achieve absorption. Such drugs are found in many therapeutic classes.

Many of the known Class II drugs are hydrophobic, and have historically been difficult to administer. Moreover, because of the hydrophobicity, there tends to be a significant variation in absorption depending on whether the patient is fed or fasted at the time of taking the drug. This in turn can affect the peak level of serum concentration, making calculation of dosage and dosing regimens more complex.

Class II drugs include itraconazole and its relatives, fluoconazole, terconazole, ketoconazole, and saperconazole; Class II anti-infective drugs, such as griseofulvin and related compounds such as griseoverdin; some anti malaria drugs (e.g. Atovaquone); immune system modulators (e.g. cyclosporine); and cardiovascular drugs (e.g. digoxin and spironolactone); and ibuprofen. In addition, drugs such as Danazol, carbamazepine, and acyclovir may also be used.

Class III drugs are biologic agents that have good water solubility and poor GI permeability including: proteins, peptides, polysaccharides, nucleic acids, nucleic acid oligomers and viruses. Examples of Class III drugs that may be used include Neomycin B, Captopril, Atenolol, and Caspofungin.

Class IV drugs are lipophilic drugs with poor GI permeability. Examples of Class IV drugs that may be used include Clorothiazide, Tobramycin, Cyclosporin, Tacrolimus, and Paclitaxel. Both Class III and IV drugs are often problematic or unsuitable for sustained release or controlled release. Class III and Class IV drugs are characterized by insolubility and poor biomembrane permeability and are commonly delivered parenterally. Traditional approaches to parenteral delivery of poorly soluble drugs include using large volumes of aqueous diluents, solubilizing agents, detergents, non-aqueous solvents, or non-physiological pH solutions. These formulations, however, can increase the systemic toxicity of the drug composition or damage body tissues at the site of administration. In one embodiment, one or more Class I, II, III, or IV drugs are included in a core of a solid oral dosage formulation, and the core is surrounded on at least its circumference by one or more bioadhesive polymers.

In a preferred method for imaging, a radiopaque material such as barium is coated with a bioadhesive material. Radioactive materials or magnetic materials could be used in place or, or in addition to, the radiopaque materials.

b. Tablets

The bioadhesive polymer may be used as one or more layers in a bioadhesive drug delivery tablet formulation. In the preferred embodiment, the formulation is a rate controlled oral dosage formulation (also referred to herein as “BIOROD”) in the form of a tablet. The bioadhesive drug delivery formulation contains a core, a bioadhesive coating, and optionally an enteric or non-enteric coating. The core contains one or more drugs, either alone or with a rate controlling membrane system. The core is enveloped on its circumference by a bioadhesive coating. FIGS. 3-11 illustrate a bioadhesive rate controlled oral dosage formulation (11), which contains at least a bioadhesive polymer (12) and a core (14).

The overall shape of the device has been designed to be compatible with swallowing. As shown in FIG. 3, the core (14) is longitudinally compressed to form a capsule-shaped tablet, which is surrounded on its circumference by a bioadhesive polymeric cylinder (12).

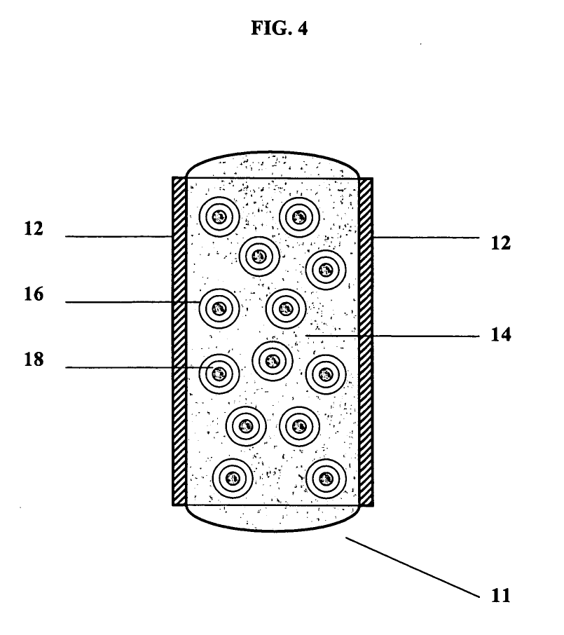

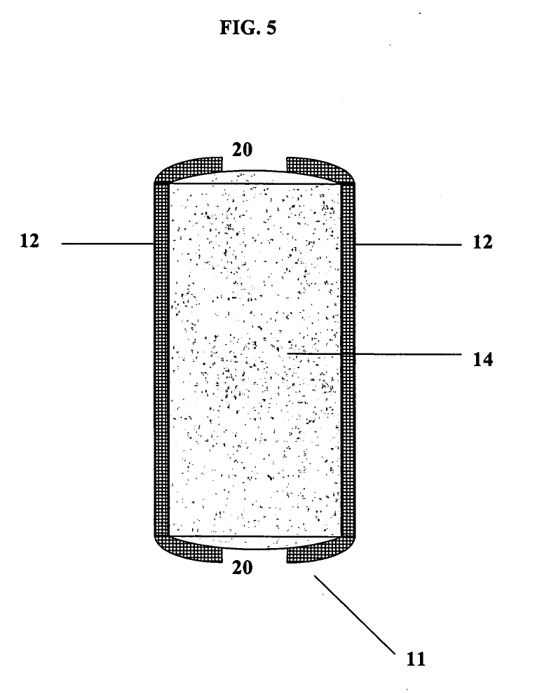

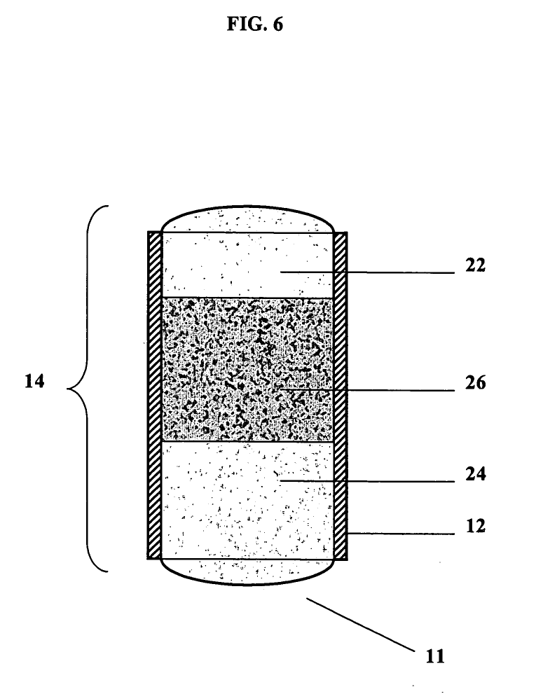

In one embodiment shown in FIG. 4, the active agent is in the form of microparticles (16), optionally the microparticles are coated with rate controlling polymers (18). In another embodiment shown in FIG. 5, the core (14) is encapsulated in a bioadhesive polymeric cylinder (12), where the cylinder contains restricted release openings at the top and bottom of the cylinder (20).

In yet another embodiment shown in FIG. 6, the core contains multiple drug layers (22 and 24). Optionally, one or more of the drug layers is a controlled release layer, one or more of the layers are immediate release layers, or one of the layers is a controlled release layer while the other layer is an immediate release layer. The tablet also contains a third drug layer (26) or a separating layer (26). Optionally, the capsule also contains restricted release openings (not shown in figure).

In another embodiment, the capsule is an osmotic drug delivery system. The entire device is coated with a semipermeable membrane, in the preferred embodiment, the membrane is a rigid semipermeable membrane.

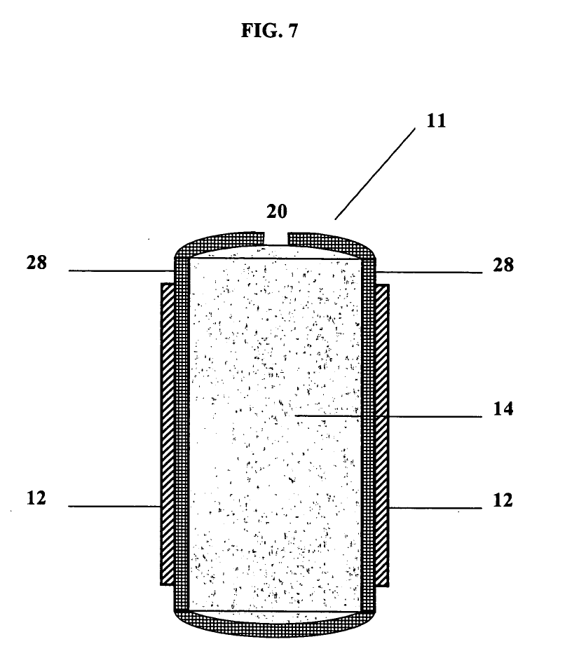

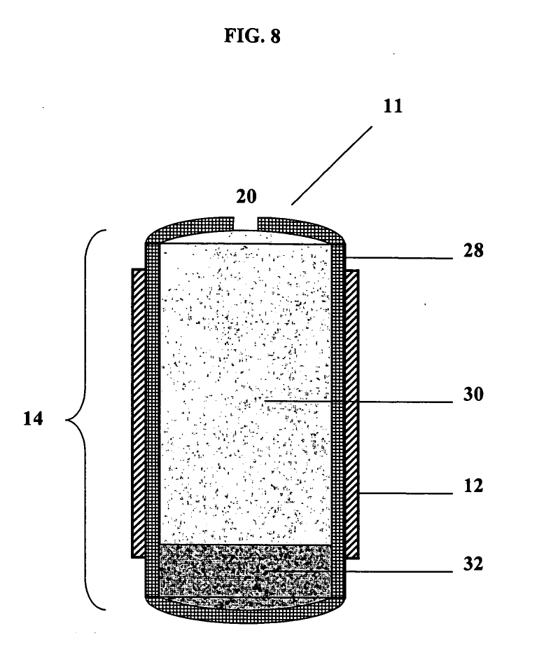

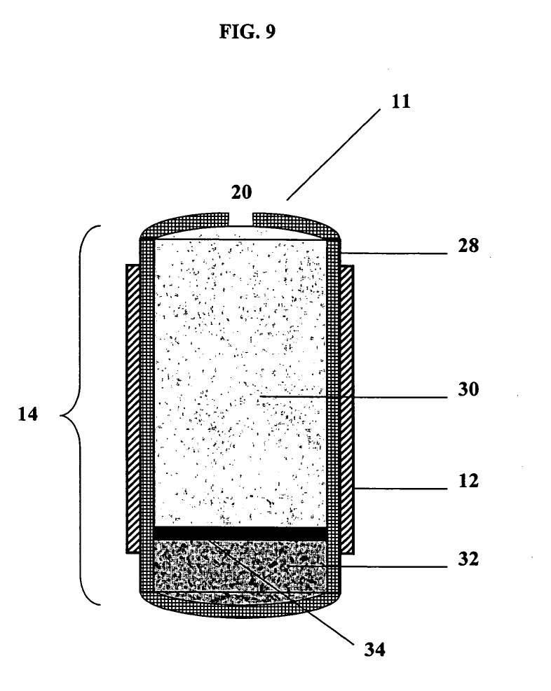

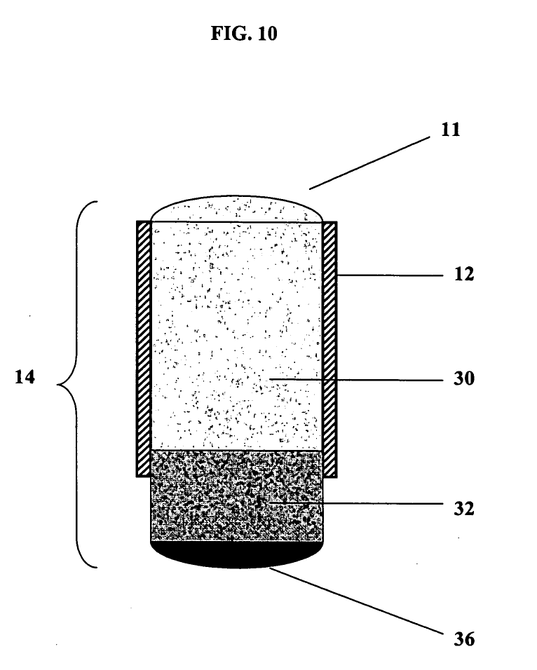

As shown in FIG. 7, an osmotic BIOROD system contains a core (14), a semi-permeable coating (28) and a bioadhesive polymer cylinder (12). The semipermeable membrane is located between the core and the bioadhesive layer. The core contains one or more drugs and osmotic agents which pull water across the semi-permeable membrane. Optionally, the capsule contains one or two restricted release openings (20) at the top and/or bottom of the bioadhesive cylinder. In the preferred embodiment, the osmotic delivery system is a “push-pull” system. Examples of this system are illustrated in FIGS. 8-10. The upper chamber contains the drug and is connected to the outside environment via a small exit hole. The lower chamber contains a swellable polymer and an osmotic attractant and may have no exit hole. Suitable osmotic agents include sugars and glycols. Once the tablet has been swallowed, water is drawn into both the upper and lower chambers. Because the lower chamber has no exit hole it expands, pushing the drug layer into the upper chamber, optionally by pushing a plug or diaphragm layer which is located between the drug layer and the push layer. Thus, the drug in the upper chamber is pushed out from the exit hole. As illustrated in FIG. 8, the core contains one layer with an active agent (30), and a second layer with a swellable polymer and osmotic agents (32). The polymer layer (32) is a “push layer” since it pushes drug out of the device when it swells at controlled rates. The system may contain at least one opening (20), as shown in FIG. 8. Optionally, the active agent (30) is separated from the push layer (32) by an insoluble plug (34) (see FIG. 9). In yet another embodiment illustrated in FIG. 10, the push-pull osmotic delivery system contains an active agent (30) in the drug layer and a swellable polymer and osmotic attractant (32) in the push layer. The drug layer (30) is surrounded on its circumference by a bioadhesive cylinder (12). The lower end of the push layer (32) is adjacent to an insoluble plug (36).

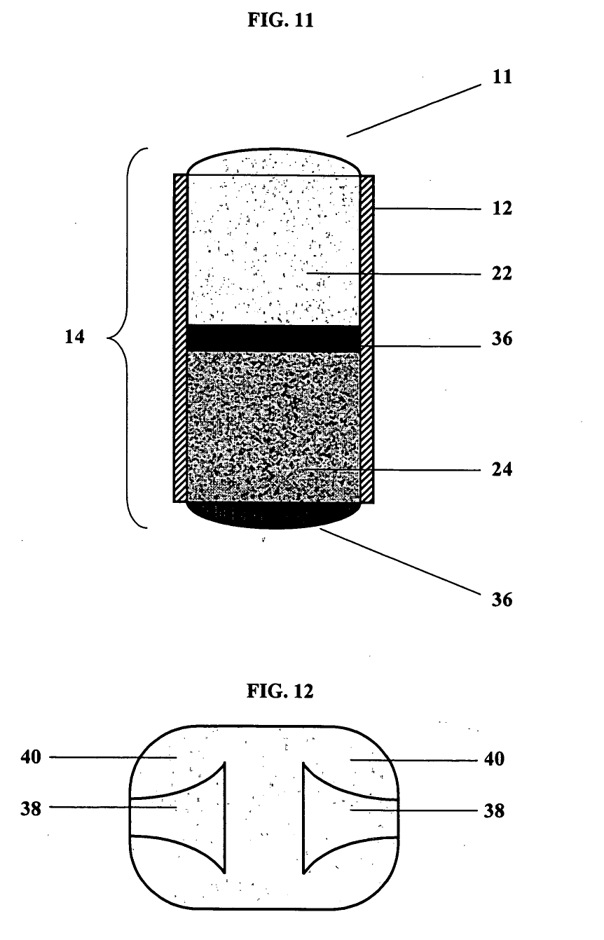

A two-pulse BIOROD system contains either the same drug in controlled release and immediate release layers in a capsule or two different drugs in either controlled release or immediate release layers in the same capsule. One embodiment of a two-pulse BIOROD system is illustrated in FIG. 11, the BIOROD system contains a plug below and above (36) the lower drug layer (24), while the upper drug layer does not contain a plug above the upper drug layer (22). This allows the drug in the upper layer (22) to be released prior to the release of the drug in the lower layer (24).

In yet another embodiment of the oral dosage formulation, the tablet contains precompressed inserts of an active agent, optionally with excipients, (38) and permeation enhancers, optionally with excipients, embedded in a matrix of bioadhesive polymer (40) (see FIG. 12). Drug is released only at the edge of the tablet and the kinetics of drug release is controlled by geometry of the inserts (38). Zero and first order release profiles are achievable with this tablet design and it is possible to have different release rates for permeation enhancer and drug by changing the configuration of the inserts.

i. Methods of Making Bioadhesive Rate Controlling Oral Dosages

The extruded bioadhesive polymer cylinder is formed of one or more bioadhesive polymers. One of the bioadhesive polymers is a biodegradable or non-biodegradable polymer backbone where a portion of the monomers that form the polymer are substituted with an aromatic group, preferably with DOPA side chains grafted onto the polymeric backbone. Other bioadhesive polymers include poly(fumaric acid- co-sebacic acid) (pFA:SA), as described in U.S. Pat. No. 5,955,096 to Mathiowitz et al. (e.g. a 20:80 copolymer of p(FA:SA)), oligomers and metal oxides, as described in U.S. Pat. No. 5,985,312 to Jacob et al., and other commercially available bioadhesive polymers, such as Gantrez (Polymethyl vinyl ether/maleic anhydride copolymers), CARBOPOL® (Noveon) (high molecular weight homo- and copolymers of acrylic acid crosslinked with a polyalkenyl polyether). Optionally the bioadhesive layer contains one or more plasticizers, pore-forming agents, and/or solvents. Suitable plasticizers include dibutyl sebacate, dibutyl adipate, dibutyl fumarate, polyethylene glycol, triethyl citrate, and PLURONIC® F68 (BASF). Suitable pore forming agents include sugars and salts, such as Sucrose, lactose, dextrose, mannitol, polyethylene glycol, sodium chloride, calcium chloride, phosphate buffer, tris buffer, and citric acid. Thermoplastic polymers can be added to the bioadhesive layer to modify the moldability and mechanical strength of the bioadhesive polymer cylinder. Suitable thermoplastic polymers include polyesters, such as poly(lactic acid-co-glycolic acid) (PLGA), poly(lactic acid) (PLA), poly(caprolactone) (PCL); methylmethacrylates, such as Eudragit RL100, Eudragit RS100, and Eudragit NE 30D; and modified celluloses, such as hydroxypropylmethylcellulose (HPMC), hydroxypropylcellulose (HPC), cellulose acetate, and ethyl cellulose.

1. Method for Production of the Hollow Bioadhesive Cylinder

In the preferred embodiment, the extruded polymer cylinder is prepared via hot-melt extrusion process, where the desired bioadhesive polymer is fed into the extruder as a pellet, flake, or powder, optionally along with one or more plasticizers. The materials are blended as they are propelled continuously along a screw through regions of high temperature and pressure to form the polymer extrudate. The extrudate is pushed from the extruder through a die having the desired shape and dimension to form a cylinder. The cylinder is cooled after extrusion. The dimensions of the cylinder can be varied to accommodate the core. The inner diameter of the cylinder can be configured to conform to the desired circumferential dimension of the preformed, pre-pressed core, which contains the therapeutic agent(s). The thickness of the cylinder is determined in part by the polymer/plasticizer type as well its behavior with respect to the external fluid. The bioadhesive nature of the polymer cylinder may also be controlled by mixing different type of polymers and excipients. Inorganic metal oxides may be added to improve the adherence. Pore formers may also be added to control its porosity. Drugs may also be added into the polymer cylinder either as a plasticizer or pore-forming agent. Adding drug to the bioadhesive layer is commonly used to increase porosity (pore-former). Some drugs are small molecules that act as plasticizers. For example, L-DOPA can behave as a plasticizer for L-DOPA-BMA.

Prior to hot-melt extrusion of the hollow cylinder, the bioadhesive polymer, optionally along with a plasticizer in a range from 0.1 to 50% (w/w), preferably 20% (w/w), is mixed in a planetary mixer. Extrusion is performed using either any standard extruder, such as MP 19 TC25 laboratory scale co-rotating twin screw extruded of APV Baker (Newcastle-under-Lyme, UK) or a Killion extruder (Killian extruder Inc., Cedar Grove, N.J.). The extruder is typically equipped with a standard screw profile with two mixing sections, an annual die with metal insert for the production of the cylinder and twin screw powder feeder. Typical extrusion conditions are: a screw speed of 5 rpm, a powder feed rate of 0.14 kg/hr and a temperature profile of 125-115-105-80-65° C. from the powder feeder towards the die. The cylinders (typically with an internal diameter of 7 mm and a wall thickness of 1 mm) are typically cut into 1 cm long cylinders.

2. Method for Production of the Inner Core System

Inner longitudinally compressed core tablets containing the therapeutic agent, and optionally other components, are compressed onto a single or multilayer tableting machine equipped with deep fill or regular tooling. For example, the therapeutic agent, either alone or in combination with a rate controlling polymer and optionally other excipients, is mixed by stirring, ball milling, roll milling or calendaring, and pressed into a solid having dimensions conforming to an internal compartment defined by the extruded polymer cylinder. One or more layers containing different therapeutic agents can be included as a multilayer tablet. The core may be a pre-fabricated insert with a semi-permeable layer on the outside of the core to form an “osmotic system” which is inserted into the bioadhesive cylinder with orifices aligned along the open ends of the cylinder.

3 Method of Insertion of the Core into the Bioadhesive Cylinder

The core, which is preferably in the form of a longitudinally compressed tablet, is inserted into the cylinder and the core and the cylinder, which forms the outer coating, are fused together to produce a solid oral dosage form. The preformed inner core with a diameter slightly smaller than the inner diameter of the cylinder is either manually or mechanically inserted into the cylinder and heated to fuse the two units. Alternately, the core insertion into the cylinder may also be done by a positive placement core insertion mechanism on the tableting machine. Initially, the extruded cylinder may be placed into the die of the machine followed by insertion of the compressed core into the internal compartment of the cylinder and the two components compressed to get the finished dosage form. Alternatively, the dosage form is prepared via simultaneous extrusion of the bioadhesive cylinder and expandable inner composition using an extruder capable of such an operation.

c. Administration of Bioadhesive Materials to Patients

The bioadhesive materials may be administered as dry powders in a suspension or in an ointment to the mucosal membranes, via the nose, mouth, rectum, or vagina. Pharmaceutically acceptable carriers for oral or topical administration are known and determined based on compatibility with the polymeric material. Other carriers include bulking agents such as METAMUCIL™. The bioadhesive material may be in a matrix or form a coating in a drug or diagnostic composition which may be administered to a patient variety of methods, including transdermal, oral, subcutaneous, intramuscular, intraperitoneal, and intravitreal administration. The material may be administered via inhalation, optionally to deliver the drug formulation to the deep lung.

The bioadhesive material may be used as an adhesive, such as a dental adhesive, a bone cement or glue, a synthetic skin or a wound dressing, a skin plaster or film. These materials may be applied directly to the site in need of treatment.

In one embodiment, the bioadhesive material is a layer in an oral dosage formulation, such as a tablet, optionally a controlled release oral dosage formulation. A patient swallows the oral dosage formulation.

These bioadhesive materials are especially useful for treatment of inflammatory bowel diseases such as ulcerative colitis and Crohn's disease. In ulcerative colitis, inflammation is restricted to the colon, whereas in Crohn's disease, inflammatory lesions may be found throughout the gastrointestinal tract, from the mouth to the rectum. Sulfasalazine is one of the drugs that is used for treatment of the above diseases. Sulfasalazine is cleaved by bacteria within the colon to sulfapyridine, an antibiotic, and to 5-amino salicylic acid, an anti-inflammatory agent. The 5-amino salicylic acid is the active drug and is active locally. Direct administration of the degradation product (5-amino salicylic acid) may be more beneficial. A bioadhesive drug delivery system can improve the therapy by retaining the drug for a prolonged time in the intestinal tract. For Crohn's disease, retention of 5-aminosalicylic acid in the upper intestine is of great importance; since bacteria cleave the sulfasalazin in the colon, the only way to treat inflammations in the upper area of the intestine is by local administration of 5-aminosalicylic acid.

Gastrointestinal Imaging Barium sulphate suspension is the universal contrast medium used for examination of the upper gastrointestinal tract, as described by D. Sutton, Ed., A Textbook of Radiology and Imaging, Vol. 2, Churchill Livingstone, London (1980), even though it has undesirable properties, such as unpalatability and a tendency to precipitate out of solution. Several properties are critical: (a) Particle size: the rate of sedimentation is proportional to particle size (i.e., the finer the particle, the more stable the suspension; (b) Non-ionic medium: charges on the barium sulphate particles influence the rate of aggregation of the particles, aggregation is enhanced in the presence of the gastric contents; (c) Solution pH: suspension stability is best at pH 5.3. However, as the suspension passes through the stomach, it is inevitably acidified and tends to precipitate. The encapsulation of barium sulfate in microspheres of appropriate size provides a good separation of individual contrast elements and may, if the polymer displays bioadhesive properties, help in coating, preferentially, the gastric mucosa in the presence of excessive gastric fluid. With bioadhesiveness targeted to more distal segments of the gastrointestinal tract, it may also provide a kind of wall imaging not easily obtained otherwise.

The double contrast technique, which utilizes both gas and barium sulphate to enhance the imaging process, especially requires a proper coating of the mucosal surface. Air or carbon dioxide must be introduced to achieve a double contrast. This is typically achieved via a nasogastric tube to provoke a controlled degree of gastric distension. Studies indicate that comparable results may be obtained by the release of individual gas bubbles in a large number of individual adhesive microspheres and that this imaging process may be used to image intestinal segments beyond the stomach.

### EXAMPLES

### Example 1

### Comparison of Tensile properties for Maleic anhydride Copolymers with and without L-DOPA

Materials: Stock polymers were prepared in-house (anhydrides) or were purchased from standard commercial sources. Several different polymers containing maleic anhydride linkages were obtained from Polysciences (CAS #'s 25655-35-0, 9006-26-2, 25366-02-8, 9011-13-6, 24937-72-2). The repeating backbone group has a different structure in each of the four polymers tested, as shown (above) in Reaction 1. Four different backbone structures were used. These are the 1:1 random copolymers of maleic anhydride with ethylene, vinyl acetate, styrene, and butadiene. The variable portions of the backbone structures are designated as R groups shown at the bottom of Reaction 1.

Methods: The polymers were grafted with L-DOPA using the route shown schematically in Reaction 1. First, the polymers were dissolved in DMSO, and L-DOPA was added to the solution. The reaction was conducted by gentle heating (70° C.) for 2 hours. During the reaction, the L-DOPA slowly went into solution. After the reaction, the reaction mixture gelled upon cooling to room temperature. The polymer was recovered by extracting the DMSO from the gel with several washes of methylene chloride. The synthesized polymers were dried and stored. Polymers were made with about 50% and about 95% molar substitution of the maleic anhydride groups with DOPA.

The polymers were dissolved in methanol for testing, for example in the texture tester described below.

Testing: The polymers described above were tested on a Texture Technologies texture analyzer machine, capable of testing material deposited as either a spray coating or a melt coating. Polymers were either melt cast onto an acorn nut or were dissolved in a solvent, preferably at 3% w/w, and sprayed onto a nylon acorn nut. An “acorn nut” is a rounded cap nut that has female threads in order to cover the end of a screw.

Solutions were sprayed through a Spraying Systems nozzle setup (SU1-SS) with a gravity feed. The polymer solution reservoir was set 6″ above the nozzle to create an inlet liquid feed of 29 mL/min. The atomization gas (nitrogen) was fed at 10 psi. The nozzle was set to oscillate horizontally such that the spray covered a 4″ span every second. Nylon acorn nuts with a head diameter of 10.8 mm (small parts, U-CNN-1420) were set, six at a time, perpendicularly along a rotating shaft (30 rpm) within the sweep area of the nozzle spray. The acorn nuts were sprayed in batches of 3 g of polymer per 6 acorn nuts. Alternatively, the acorn nuts were coated by dipping them into a molten polymer or a concentrated polymer solution. The acorn nuts were singly tested on the texture analyzer and brought into contact with the mucosal side of a flattened section of pig jejunum at a rate of 0.5 mm/second and an applied force of 5 g. The acorn nut was held at this position for 420 seconds and then pulled away at a rate of 0.5 mm/second. The force as a function of distance was plotted on an output graph. The fracture strength and tensile work were calculated form the output graph and corrected for the projected acorn nut surface area.

In a variant useful for testing underwater adhesion (the “Wet Method”). samples were tested in a small aquarium where the pig jejunum tissue was anchored to the bottom of the aquarium, and the coated acorn nuts were brought into contact with the tissue while both were submerged in phosphate buffered saline (PBS, pH=7.3-7.5).

Results: Tensile properties were tested on three polymer preparations and a mixture (control), and are shown in Table 1. The mixture (top line) was a 2:1 (w/w) mixture of polycaprolactone (MW 1,000,000; from Scientific Polymer Products) with L-DOPA. (Note that no reaction was anticipated or observed between DOPA and polycaprolactone.) A first polymer (control) was butadiene-co-maleic anhydride at a 1:1 ratio, with no added DOPA. The other two were the same backbone polymer, with a nominal 50% substitution of the maleic anhydride with DOPA, and a nominal 100% substitution of the maleic anhydride with DOPA. Actual substitution was approximately 95% of the theoretical amount.

In Table 1, it can be seen that increasing substitution with DOPA increased both fracture strength and tensile work to fracture. In addition, the sample with maximal DOPA substitution had essentially the same values when tested wet and dry. (“Wet” data are not shown.)

TABLE 1
Substitution, Fracture Strength and Tensile Work of L-DOPA
grafted Polymers

| | Maleic | | | |
|---|---|---|---|---|
| | Anhydride | | | |
| | Grafted w/L- | | | |
| | DOPA | | | |
| | (mol L- | | Fracture | Tensile |
| | DOPA/mol | Test | Strength | Work |
| Polymer | Anhydride) | Method | (mN/cm²) | (nJ) |
| Polycaprolactone | n/a | Dry | 491 | 69,400 |
| blend L-DOPA | | | | |
| (2:1) | | | | |
| Poly [butadiene- | 0% | Dry | 173 | 14,750 |
| co-maleic | | | | |
| anhydride] | | | | |
| (50:50) | | | | |
| Poly [butadiene- | 50% | Dry | 214 | 18,250 |
| co-maleic | | | | |
| anhydride] | | | | |
| (50:50) | | | | |
| Poly [butadiene- | 95% | Dry | 346 | 84,200 |
| co-maleic | | | | |
| anhydride] | | | | |
| (50:50) | | | | |

To better understand this observation, the three butadiene-maleic anhydride polymers used in Table 1 were compared with a preparation of a 2:1 mixture of a grade of EUDRAGIT™, a polyacrylate polymer (type RL100, 150,000 MW), with an oligomer of fumaric anhydride (FA) (200-400 MW), optionally containing calcium oxide (CaO) in an amount of 25% by weight of the formulation CaO as an enhancer of adhesion.

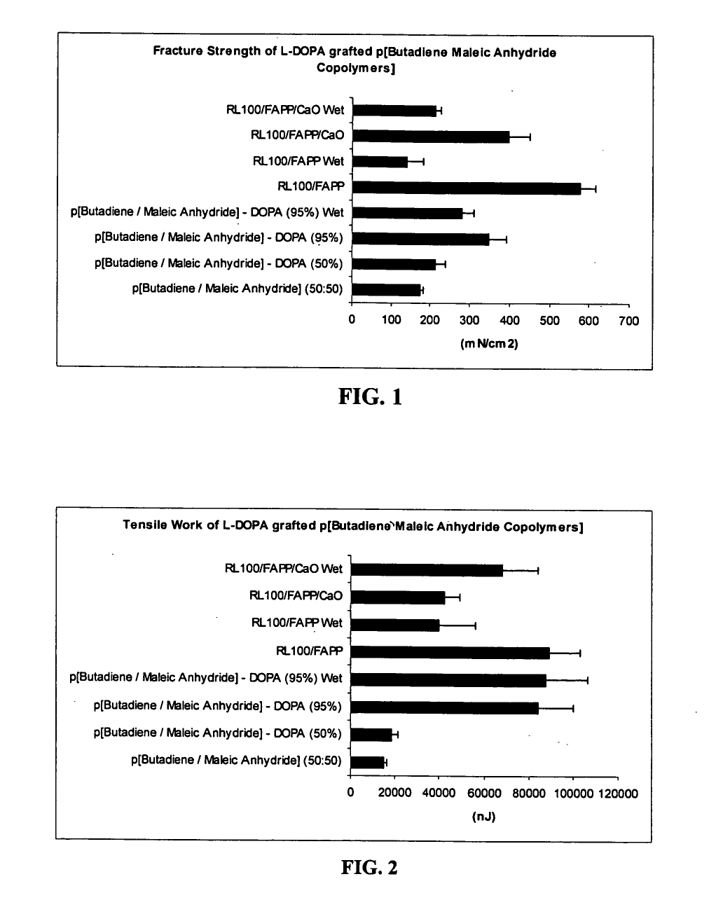

FIG. 1 compares the fracture strength of these materials both wet and dry. It can be seen that while the 95%-DOPA grafted anhydride was not the strongest polymer when dry when compared with RL100/FAPP and RL100/FAPP/CaO, it was the strongest when wet. On wetting, it lost only about 20% of its fracture strength, while the RL100/FAPP preparation lost over 75% of its tensile strength, and the RL100/FAPP/CaO lost over 50% of its tensile strength when wet.

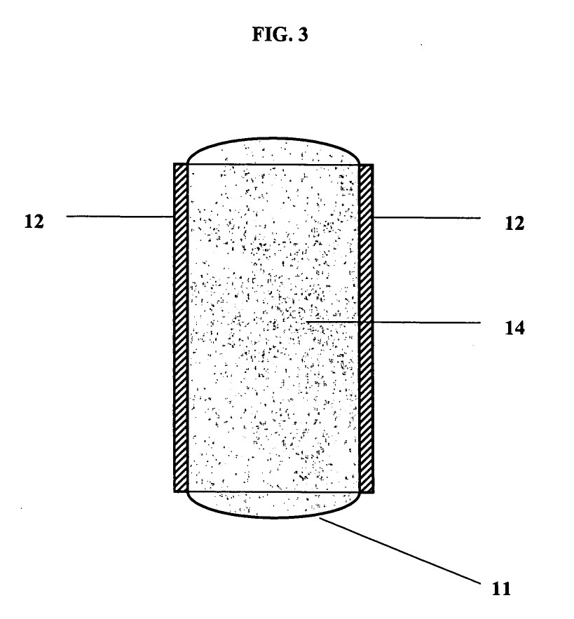

FIG. 2 graphically depicts the amount of tensile work required to fracture the compositions. In this test, the highly DOPA-substituted polymer had values comparable to those of the RL100/FAPP material, but the DOPA polymer improved when wet while the RL100/FAPP preparation declined dramatically. The RL110/FAPP/CaO was worse when dry, but increased the most when wet.

Butadiene was preferable as a backbone for an adhesive. It is possible that this is because butadiene provides a rigid spacer between maleic anhydride groups, allowing the reaction to occur with less steric hindrance. Bulky groups such as styrene may cause steric hindrance preventing complete substitution of L-DOPA groups. Likewise, ethylene groups may prevent the reaction from going to completion due to hindrance from the close proximity of already reacted L-DOPA groups.

Examples 2-5 describe studies using to L-DOPA-Butadiene maleic anhydride (BMA) polymer formulated as adhesive outer layers in a tablet designed for oral administration. The L-DOPA-BMA polymer has a weight average molecular weight of about 15 kDa), where about 95% of the monomers were substituted with L-DOPA (also known as Spheromer III™ Bioadhesive polymer, Spherics, Inc.). FIG. 3 illustrates one embodiment of the tablet.

### Example 2

### Fluoroscopy Study of Barium-Impregnated Trilayer Tablets with Bioadhesive Polymer Outer Layers

Method of Manufacture: Trilayer tablets were prepared by sequentially filling a 0.3287×0.8937 “00 capsule” die (Natoli Engineering) with 333 mg of L-DOPA-Butadiene maleic anhydride (Weight average molecular weight of about 15 kDa), where about 95% of the monomers were substituted with L-DOPA (also known as LDOPA-BMA or Spheromer III™ Bioadhesive polymer, Spherics, Inc.) to form a first outer layer, followed by 233 mg of a blend of hydroxypropylmethylcellulose (HPMC) with a viscosity of 4000 cps and 100 mg of barium sulfate to form the inner layer, followed by an outer layer of 333 mg of LDOPA-BMA. Trilayer tablets were prepared by direct compression at 2000 psi for 1 second using a Globepharma Manual Tablet Compaction Machine (MTCM-1).

Testing: The tablets were administered to female beagles that were fasted for 24 hours (fasted). The tablets were also dosed to fasted beagles that had been fed with chow, 30 minutes prior to dosing (fed). Tablets were continuously imaged with fluoroscopy over the course of 6 hours in unrestrained dogs.

Results: Trilayer tablets with Spheromer III in the bioadhesive layers remained in the stomach of fasted dogs for up to 3.5 hours and resided in the stomach of fed dogs in excess of 6 hours. The tablets did not mix with food contents and remained in contact with stomach mucosa at the same location until they passed into the small intestine.

### Example 3

### Comparison of SPORANOX®, Spherazole™ IR and Spherazole™ CR Tablets

Spherazole™ IR is an immediate release formulation of itraconazole that has lower variability than the innovator product, SPORANOX®. The drug substance itraconazole is spray-dried with Spheromer I bioadhesive polymer to reduce drug particle size and blended with excipients including croscarmellose (superdisintegrant), talc (glidant), microcrystalline cellulose (binder/filler) and magnesium stearate (lubricant). The blend is dry granulated by slugging, to increase bulk density, and subsequently milled, sieved and compressed. The final product is a 900 mg oval tablet containing 100 mg of itraconazole, identical to the Sporonox dose. The composition of the tablet is 11% itraconazole; 14.8% Spheromer I; 11.1% HPMC 5 cps (E5), 2% Talc, 19.7% Cross-linked carboxymethylcellulose sodium (AcDiSOL), 1% Magnesium Stearate, and 40.3% Microcrystalline cellulose. When tested in the “fed” beagle model, the IR formulation has an AUC in the range of 20,000±2000 ng/ml*hr-1, Cmax of 1200±ng/ml, tmax of 2±1 hrs. This performance is equivalent to performance of Sporonox in the fed dog model and less variable than the innovator product.

By comparison, Spherazole CR is formulated as a controlled release tablet. Itraconazole is dissolved in solvent with Eudragit E100 and either spray-dried or drug-layered onto MCC cores, blended with HPMC) of different viscosities (5, 50, 100, 4000 cps) and other excipients (corn starch, lactose, microcrystalline cellulose or MCC) to control drug release. The rate controlling inner drug layer is then sandwiched between outer adhesive layers composed of Spheromer I or III and optionally Eudragit RS PO to improve mechanical properties of the bioadhesive layer. Spherazole CR when tested in the fed beagle model has AUC in the range of 20,000±2000 ng/ml*hr-1, Cmax of 600±ng/ml, tmax of 8-20 hrs depending on the particular composition of the rate-controlling core. The performance of the CR product is similar to Spherazole IR and Sporanox with respect to AUC, however, Cmax is lower by 50%, an important benefit in terms of reduced side effects and drug toxicity. The extended tmax facilitates qd dosing compared to bid dosing for the innovator and IR products.

### Example 4

### Bioadhesive Controlled Release Trilayer Tablet with 100 mg Spray-Dried Itraconazole

Trilayer tablets were prepared as described in Example 1, using the formulation listed below and were tested once (n=6/test) in the fed beagle model. The tablets contained an inner core (333 mg) containing 100% w/w of Itraconazole spray-dried with a low viscosity hydroxypropylmethylcellulose, HPMC E5 (5 cps viscosity), forming a 30% (w/w) itraconazole spray dried composition. The tablets contained an outer layer (formed of two 333 mg compositions). The outerlayer (333 mg×2) contained 66% w/w Spheromer III, 33% w/w Polyplasdone XL (Crospovidone), and 1% w/w Magnesium Stearate.

The AUC of the CR formulation was similar to the AUC range for Immediate Release Itraconazole Tablet and SPORANOX® (Johnson & Johnson) in the same fed beagle model. Immediate Release Itraconazole Tablet is an immediate release formulation of itraconazole that has lower variability than the brand name formulation, SPORANOX®. As shown in FIG. 12, the AUC for the controlled release itraconazol tablet was 20.971 ng/(mL*hr), Cmax was 602 ng/mL and Tmax was 29 hrs.

### Example 5

### Comparison of Three Controlled Release Tablets Containing 400 mg of Acyclovir, Two Bioadhesive and One-Non-Adhesive, Versus Zovirax Tablet (400 mg)

Tablets

Tablet 1 (Lot 404-093) was prepared with a core (539 mg) containing 74% w/w Acyclovir (400 mg), 12.4% w/w HPMC 100 cps, 6.2% w/w HPMC 5 cps, 3.1% w/w Glutamic Acid (acidulant), 3.1% w/w Corn Starch 1500, and 0.7% w/w Magnesium Stearate, and an outer bioadhesive layer containing (250 mg×2) 99% w/w Spheromer III and 1% w/w Magnesium Stearate.

Tablet 2 (Lot 404-134) was prepared with a core (600 mg) containing 67.6% w/w Acyclovir (400 mg), 16.9% w/w Ethocel 10 Standard FP, 11.3% w/w Glutamic Acid (acidulant), 2.7% w/w Talc, 0.5% w/w Aerosil 200, and 1.0% w/w Magnesium Stearate and with an outer layer containing (300 mg×2) 99% w/w Spheromer III and 1% w/w Magnesium Stearate.

Tablet 3 (Lot 404-182) is the same as Tablet 1, except that Spheromer III is replaced with non-adhesive polyethylene in the outer bilayer.

In Vitro Dissolution data

The three controlled release tablets were each tested for dissolution in SGF, pH 1.2 in a USP 2 Paddle apparatus at 100 rpm.

TABLE 2
In Vitro Dissolution Data for Tablet 1

| Time | Tablet 1 |
|---|---|
| (min) | (% Release) |
| 0 | 0 |
| 10 | 5.3 |
| 30 | 12.9 |
| 60 | 29.3 |
| 120 | 55.4 |
| 180 | 75.4 |
| 270 | 90.5 |

TABLE 3
In Vitro Dissolution Data for Tablet 2

| Time | Tablet 2 |
|---|---|
| (min) | (% Release) |
| 0 | 0 |
| 10 | 3.3 |
| 30 | 7.1 |
| 60 | 11.3 |
| 120 | 20.3 |
| 180 | 27.3 |
| 270 | 37.8 |

Pharmacokinetic Profiles for Tablets

A single 400 mg dose of each Tablet was administered to 6 beagle dogs in the “fed” state and the following pharmacokinetic profiles resulted. These profiles are compared to ZOVIRAX® (Glaxo Wellcome Inc.) tablet (400 mg), the brand name oral acyclovir formulation. Each 400-mg tablet of ZOVIRAX® contains 400 mg of acyclovir and the inactive ingredients magnesium stearate, microcrystalline cellulose, povidone, and sodium starch glycolate.

TABLE 4
ZOVIRAX ® tablet (400 mg) Plasma Acyclovir (mg/ml)

| | Mean | SD | SE |
|---|---|---|---|
| 0 hr. | 0.0 | 0.0 | 0.0 |
| 0.5 hr. | 8.6 | 5.3 | 2.4 |
| 1 hr. | 14.2 | 4.5 | 2.0 |
| 1.5 hr. | 21.0 | 8.0 | 3.6 |
| 2 hr. | 17.4 | 5.2 | 2.3 |
| 2.5 hr. | 17.5 | 8.8 | 3.9 |
| 4 hr. | 7.9 | 2.5 | 1.1 |
| 6 hr. | 4.1 | 1.5 | 0.7 |
| 8 hr. | 2.3 | 0.7 | 0.3 |
| 10 hr. | 2.0 | 1.3 | 0.6 |
| 12 hr. | 2.6 | 2.9 | 1.3 |
| 24 hr. | 0.2 | 0.2 | 0.1 |
| AUC | 97.7 | 30.3 | 13.6 |
| Cmax | 22.6 | 7.7 | 3.4 |
| Tmax (hr.) | 1.6 | 0.8 | 0.4 |

TABLE 5
Tablet 1 Plasma Acyclovir (mg/ml)

| | mean | sd | se |
|---|---|---|---|
| 0 hr. | 0.0 | 0.0 | 0.0 |
| 0.5 hr. | 2.1 | 1.4 | 0.7 |
| 1 hr. | 6.6 | 2.2 | 1.1 |
| 1.5 hr. | 8.5 | 2.6 | 1.3 |
| 2 hr. | 10.4 | 3.2 | 1.6 |
| 2.5 hr. | 12.3 | 3.1 | 1.5 |
| 4 hr. | 12.7 | 4.7 | 2.3 |
| 6 hr. | 9.0 | 3.9 | 2.0 |
| 8 hr. | 5.0 | 1.9 | 1.0 |
| 10 hr. | 2.6 | 1.1 | 0.5 |
| 12 hr. | 2.2 | 1.2 | 0.6 |
| 24 hr. | 0.2 | 0.1 | 0.0 |
| AUC | 98.0 | 28.8 | 14.4 |
| Cmax | 13.9 | 3.6 | 1.8 |
| Tmax (hr.) | 3.7 | 0.7 | 0.3 |

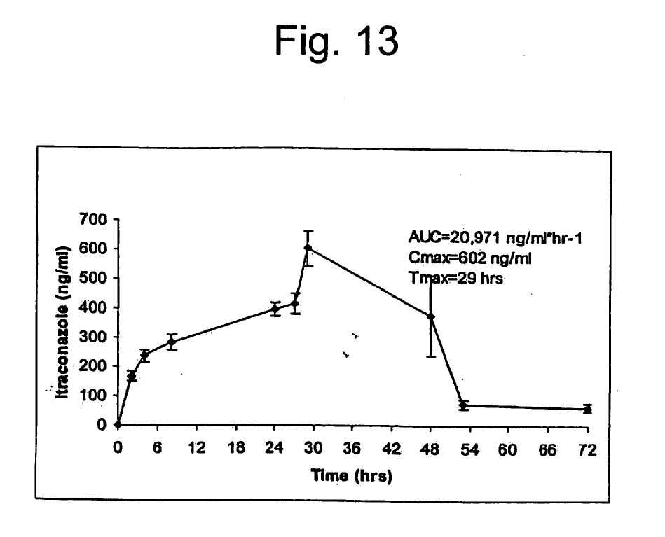

The data listed in Tables 4 and 5 and charted in FIG. 13 shows that that the AUC of Tablet 1 was nearly identical to the AUC for the Zovirax® 400 mg tablet (i.e. 98% of the AUC for the Zovirax® 400 mg tablet). As shown in FIG. 12, the Cmax was 62% of the Zovirax® 400 mg tablet, and the Tmax shifted from 1.6 hrs for Zovirax to 3.7 hrs for Tablet 1.

TABLE 6
Tablet 2 Plasma Acyclovir (mg/ml)

| | mean | sd | se |
|---|---|---|---|
| 0 hr. | 0.0 | 0.0 | 0.0 |
| 0.5 hr. | 0.3 | 0.2 | 0.1 |
| 1 hr. | 1.3 | 0.9 | 0.4 |
| 1.5 hr. | 3.0 | 2.6 | 1.2 |
| 2 hr. | 4.8 | 4.2 | 1.9 |
| 2.5 hr. | 6.8 | 5.0 | 2.3 |
| 4 hr. | 10.0 | 5.9 | 2.6 |
| 6 hr. | 10.9 | 5.0 | 2.2 |
| 8 hr. | 10.7 | 4.4 | 1.9 |
| 10 hr. | 6.9 | 4.1 | 1.8 |
| 12 hr. | 4.5 | 3.3 | 1.5 |
| 24 hr. | 0.2 | 0.2 | 0.1 |
| AUC | 118.7 | 45.0 | 20.1 |
| Cmax | 13.1 | 4.0 | 1.8 |
| Tmax (hr.) | 5.1 | 2.3 | 1.0 |

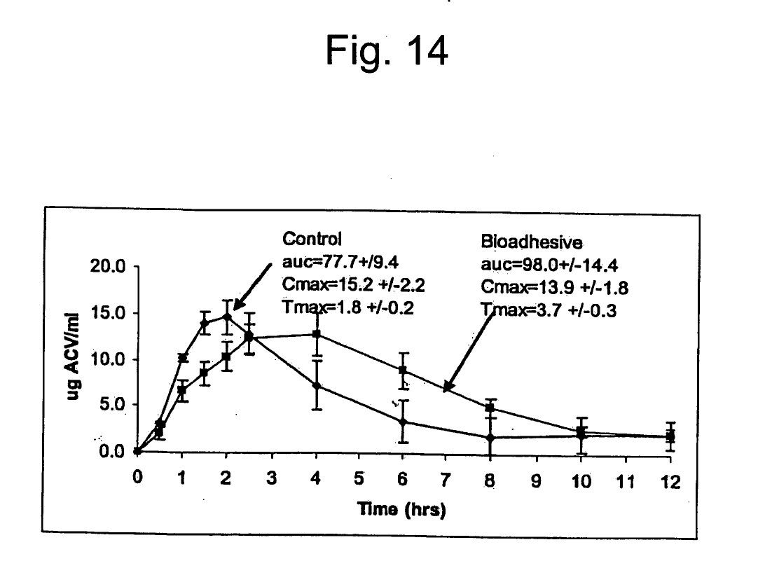

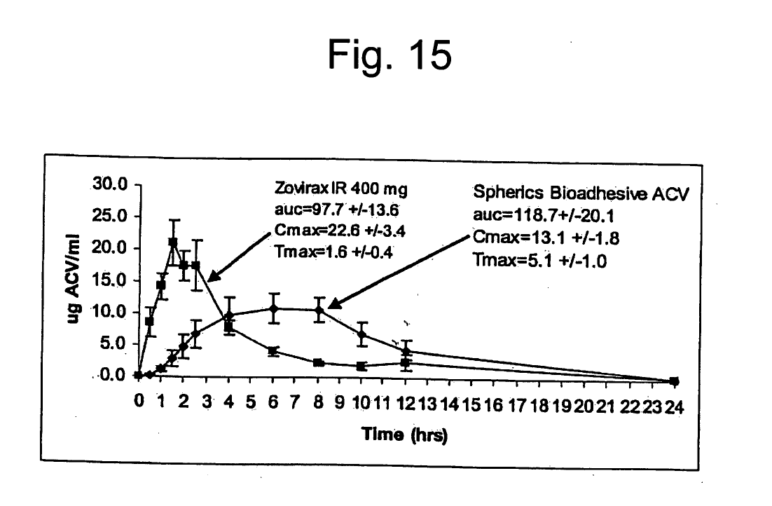

The data listed in Tables 4 and 6 and charted in FIG. 14 shows that the AUC of Tablet 2 was higher than the ZOVIRAX® 400 mg tablet, the Cmax was 58% of the ZOVIRAX® 400 mg tablet, and the Tmax shifted from 1.6 hrs for ZOVIRAX® 400 mg tablet to 5.1 hrs for Tablet 2.

TABLE 7
Tablet 3 Plasma Acyclovir (mg/ml)

| | mean | sd | se |
|---|---|---|---|
| 0 hr. | 0.0 | 0.0 | 0.0 |
| 0.5 hr. | 3.1 | 3.9 | 1.7 |
| 1 hr. | 10.2 | 7.7 | 3.4 |
| 1.5 hr. | 14.0 | 6.6 | 2.9 |
| 2 hr. | 14.6 | 4.6 | 2.1 |
| 2.5 hr. | 12.8 | 3.2 | 1.4 |
| 4 hr. | 7.3 | 1.9 | 0.9 |
| 6 hr. | 3.4 | 1.3 | 0.6 |
| 8 hr. | 2.0 | 0.4 | 0.2 |
| 10 hr. | 2.2 | 1.9 | 0.9 |
| 12 hr. | 2.2 | 2.6 | 1.2 |
| 24 hr. | 0.1 | 0.2 | 0.1 |
| AUC | 77.7 | 20.9 | 9.4 |
| Cmax | 15.2 | 5.0 | 2.2 |
| Tmax | 1.8 | 0.4 | 0.2 |

The data listed in Tables 4 and 7 shows that The AUC of the non-adhesive Tablet 3 was lower than the ZOVIRAX® 400 mg tablet, the Cmax was 67% of the ZOVIRAX® 400 mg tablet, and the Tmax was similar to the Tmax for the ZOVIRAX® 400 mg tablet.

### Example 6

### Comparison of DL-DOPA-BMA with L-DOPA-BMA

Two different compounds DOPA containing compounds were synthesized, L-3,4-dihydroxyphenylalanine (L-DOPA) and a (50:50) racemic mixture of D,L-3,4-dihydroxyphenylalanine (DL-DOPA). L-DOPA and Dl-DOPA were each grafted onto a Butadiene Maleic Anhydride backbone. Approximately 95% of the monomers were substituted with L-DOPA or DL-DOPA. The mucoadhesion of both the L-DOPA and DL-DOPA polymers was tested using a Stable Micro Systems Texture Analyzer and an experimental setup known to those skilled in the art. Six samples for each polymer were tested. The mean fracture strength of the DL-DOPA-BMA polymer was 0.0139N, with a standard deviation of 0.0090 N. The mean fracture strength of the L-DOPA-BMA polymer was 0.0134 N, with a standard deviation of 0.0042 N. The mean total tensile work for the DL-DOPA-BMA polymer was 0.0045 nJ, with a standard deviation of 0.0023 nJ. The mean tensile work for the L-DOPA-BMA polymer was 0.005 nJ, with a standard deviation of 0.0018 nJ. There was no statistical difference between either the peak detachment force or the total tensile work associated with each polymer.

### Example 7

### Comparison of the Addition of Different Plasticizers to L-DOPA-BMA Polymer Films

Plasticizers may be added to the bioadhesive polymers to improve their flexibility. The affect of different plasticizers on an L-DOPA-BMA polymer was studied. Approximately 95% of the monomers were substituted with L-DOPA.

Methods: Polymer films were prepared by dissolving 320 mg L-DOPA-BMA polymer and 80 mg plasticizer in 20 mL of methanol. These solutions were then allowed to slowly evaporate in circular Teflon coated wells overnight. After film formation, the films were removed and lyophilised for 24 hours to remove any residual methanol. The dried films were ground, and their glass transition temperature (T_{g}) was measured by differential scanning calorimetry (DSC).

Testing: All measurements were performed on a Perkin Elmer Pyris 6 DSC in Perkin Elmer aluminium plates. The following thermal program was used:

1. Isothermal: 2 minutes at 10° C.
2. Heat: 10° C. to 200° C. at 10° C. per minute
3. Cool: 200° C. to 10° C. at 10° C. per minute
4. Isothermal: 2 minutes at 10° C.
5. Heat: 10° C. to 200° C. at 10° C. per minute
   All T_{g} measurements were taken from the second heating cycle.

Results: The results for this study are summarized in Table 8.

TABLE 8
Glass Transition Temperature of L-DOPA/BMA Polymer
Films Containing a Series of Plasticizers

| Plasticizer | T_{g} (° C.) |
|---|---|
| None | 151 |
| Di-sec-butyl fumarate (DBF) | 89 |
| Diethyl phthalate (DEP) | ND |
| Dibutyl sebacate (DBS) | 96 |
| Di-iso-butyl adipate (DIA) | 95 |
| Triethyl Citrate (TEC) | 72 |
| poly(ethylene glycol) (PEG) | 74 |
| Lutrol (F-68) | 50 |

ND = None Detected

Polymer films with low glass transition temperatures are desirable for processes that involve coating a material with thin films. Polymers with high levels of crystallinity, often need a plasticizer present in these films to lower the T_{g}. As indicated in Table 8, L-DOPA/BMA polymer is a very crystalline polymer, with a high glass transition temperature of 151° C. As shown by the data in Table 8, DBF, DBS and DIA all have plasticizing effects on L-DOPA/BMA, lowering the T_{g} consistently by at least 37%. All of these plasticizers are diesters, which are water insoluble. However, the plasticizers which had the strongest affect were TEC, PEG, and F-68, which are water-soluble plasticizers. F-68 lowered the T_{g} by 67% to 50° C.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A bioadhesive material comprising a polymeric backbone and a side chain or side group containing an aromatic group substituted with one or more hydroxyl groups.

2. The material of claim 1, wherein the aromatic group is catechol.

3. The material of claim 2, wherein the aromatic group is a derivative of catechol.

4. The material of claim 3, wherein the derivative of catechol is 3,4-dihydroxyphenylalanine (DOPA).

5. The material of claim 1, wherein the polymeric backbone is a hydrophobic polymer.

6. The material of claim 5, wherein the hydrophobic polymer is selected from the group consisting of polyanhydrides, polyacrylates, polyorthoesters, polyesters, and polyhydroxy acids.

7. The material of claim 6, wherein the hydrophobic backbone is a polyanhydride.

8. The material of claim 1, wherein at least 10% of the monomers in the polymeric backbone contain a side chain containing an aromatic group.

9. The material of claim 8, wherein at least 50% of the monomers in the polymeric backbone contain a sidechain containing an aromatic group.

10. The material of claim 1, further comprising a therapeutic, prophylactic, or diagnostic agent.

11. The material of claim 9, wherein the therapeutic agent is L-3,4-dihydroxyphenylalanine (levodopa).

12. A method for forming a bioadhesive material, comprising reacting a polymer with a compound containing an aromatic group substituted with one or more hydroxyl groups or a poly(amino acid), peptide or protein containing an aromatic group substituted with one or more hydroxyl groups, wherein the a poly(amino acid), peptide or protein has a molecular weight of 20 kDa or less.

13. The method of claim 12, wherein the polymer comprises an amino reactive group and wherein the compound comprises an amino group.

14. The method of claim 12, wherein the polymer is a hydrophobic polymer.

15. The method of claim 14, wherein the hydrophobic polymer is selected from the group consisting of polyanhydrides, polyacrylates, polyorthoesters, polyesters, and polyhydroxy acids.

16. The method of claim 14, wherein the hydrophobic polymer is a polyanhydride.

17. The method of claim 11, wherein the compound comprising an aromatic group is a catechol derivative.

18. The method of claim 17, wherein the catechol derivative is 3,4-dihydroxyphenylalanine (DOPA).

19. A method for forming a bioadhesive material, comprising reacting a monomer with a compound containing an aromatic group substituted with one or more hydroxyl groups or a poly(amino acid), peptide or protein containing an aromatic group substituted with one or more hydroxyl groups, wherein the a poly(amino acid), peptide or protein has a molecular weight of 20 kDa or less, and polymerizing the monomer.

20. The method of claim 19, wherein the monomer comprises an amino reactive group and wherein the compound comprises an amino group.
